(19) United States
(12) Patent Application Publication
(10) Pub. No.: US 2025/0290126 A1
(43) Pub. Date: Sep. 18, 2025

(54) Field-Deployable Polymerase Chain Reaction Test Assembly Kit and Method of Use

(71) Applicants: William D. Bickmore, St. George, UT (US); Neil D. Holland, St. George, UT (US)

(72) Inventors: William D. Bickmore, St. George, UT (US); Neil D. Holland, St. George, UT (US)

(21) Appl. No.: 19/222,408

(22) Filed: May 29, 2025

Related U.S. Application Data
(63) Continuation of application No. 17/715,705, filed on Apr. 7, 2022, now abandoned.

(51) Int. Cl. C12Q 1/686; B01L 3/00; B01L 7/00

(52) U.S. Cl. CPC C12Q 1/686; B01L 3/50273; B01L 7/52; B01L 2300/1827; B01L 2400/0478; B01L 2400/0481

(57) ABSTRACT

A field-deployable polymerase chain reaction (PCR) test assembly enabling onsite completion of a PCR test in less than thirty minutes includes a barrel to receive a fluid. A piston is insertable into the barrel to seal the fluid within the barrel. An ultrasonic transducer is in communication with the barrel and provides transient cavitation in the fluid to release DNA or RNA from a biological sample in the fluid. A purification column is positioned within the barrel. A manifold is in fluidic communication with the barrel. A quantity of PCR reagents is positioned in a vial that is attachable to the manifold. The piston supplies pressure on the fluid to drive the fluid downwardly through the purification column and the manifold and into the vial. An optical system emits light into the vial and reads fluorescent light that indicates the presence of a target DNA within the vial.

### CROSS-REFERENCE TO RELATED APPLICATIONS

I hereby claim the benefit under 35 U.S.C., Section 120 of U.S. application Ser. No. 17/715,705 filed Apr. 7, 2022.

### (c) STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE via THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to field-deployable test assemblies and more particularly pertains to a new field-deployable test assembly enabling onsite completion of a polymerase chain reaction (PCR) test in less than thirty minutes. Conventionally extraction and purification steps for obtaining DNA or RNA from a biological sample typically must be performed in a laboratory before thermocycling. There remains a need for improved field-deployable PCR test assemblies and in particular field-deployable PCR test assemblies that are both lightweight and which can be used by technicians with limited PCR test experience while generating a reliable test result.

### (2) Description of Related Art including information disclosed under 37 CFR 1.97 and 1.98

The prior art relates to field-deployable test assemblies, but which lack both an integrated sonication capability for preparing a biological sample and a highly sensitive optical system for increased sensitivity enabling rapid completion of a polymerase chain reaction test.

### BRIEF SUMMARY OF THE INVENTION

An embodiment of the disclosure meets the needs presented above by generally comprising a barrel that is configured to receive a fluid. A piston is insertable into the barrel to seal the fluid within the barrel. An ultrasonic transducer is in communication with the barrel and is configured to provide transient cavitation pressure on the fluid to release DNA or RNA from a biological sample in the fluid. A purification column is positioned within the barrel. A manifold and a vial are in fluidic communication with the barrel and a quantity of polymerase chain reaction (PCR) reagents is positioned in the vial. The piston supplies pressure on the fluid to drive the fluid downwardly through the purification column and the manifold and into the vial. An optical system emits an excitation wavelength of light into the vial and is configured to read fluorescent light that is emitted from the vial which indicates the presence of DNA within the vial.

Another embodiment of the disclosure includes a field-deployable polymerase chain reaction test kit, which includes the barrel, the piston, the manifold, the vial in which the PCR reagents are sealed, the purification column, a sealed container containing a predetermined amount of the fluid, the ultrasonic transducer, and the optical system. The field-deployable polymerase chain reaction test kit also includes a thermocycling unit and a microprocessor. The barrel containing the purification column, the piston, the manifold, the vial containing the PCR reagents, and the sealed container are within a barrier package to maintain them in a contaminant-free environment. The PCR test kit is intended to enable onsite completion of a PCR test in less than thirty minutes.

Yet another embodiment of the disclosure includes a method for completing a PCR test in less than thirty minutes outside of a laboratory. The method entails provision of the field-deployable PCR test kit, as per the discussion above. The fluid is added to the barrel, the biological sample is added to the fluid, and the piston is inserted into the barrel. The ultrasonic transducer is attached to the barrel and is actuated to release DNA and/or RNA from the biological sample. The piston then is pushed downwardly to motivate the fluid through the purification column so that a filtrate passes to the vial. The optical system then is actuated to indicate the presence of dsDNA within the vial.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a cross-sectional view of a field-deployable polymerase chain reaction test assembly according to an embodiment of the disclosure.

FIG. 2 is a cross-sectional view of an embodiment of the disclosure.

FIG. 3 is a cross-sectional view of an embodiment of the disclosure.

FIG. 4 is a partial cross-sectional view of an embodiment of the disclosure.

FIG. 5 is a detail view of an embodiment of the disclosure.

FIG. 6 is a chart of cumulative trials of data taken from a log-based optical system for the first 10 cycles of a PCR test.

FIG. 7 is a flow diagram for a method utilizing an embodiment of the disclosure.

FIG. 8 is kit view of an embodiment of the disclosure.

### DETAILED DESCRIPTION OF THE INVENTION

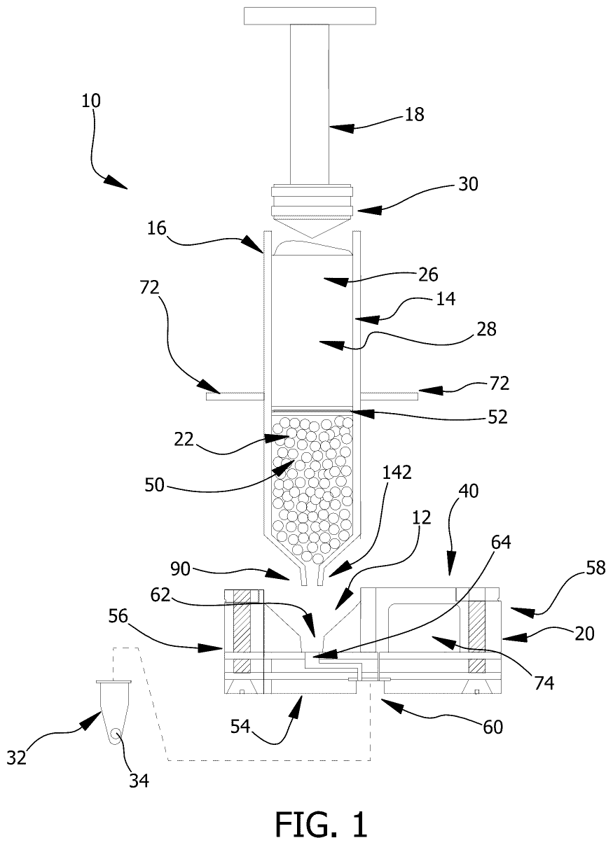

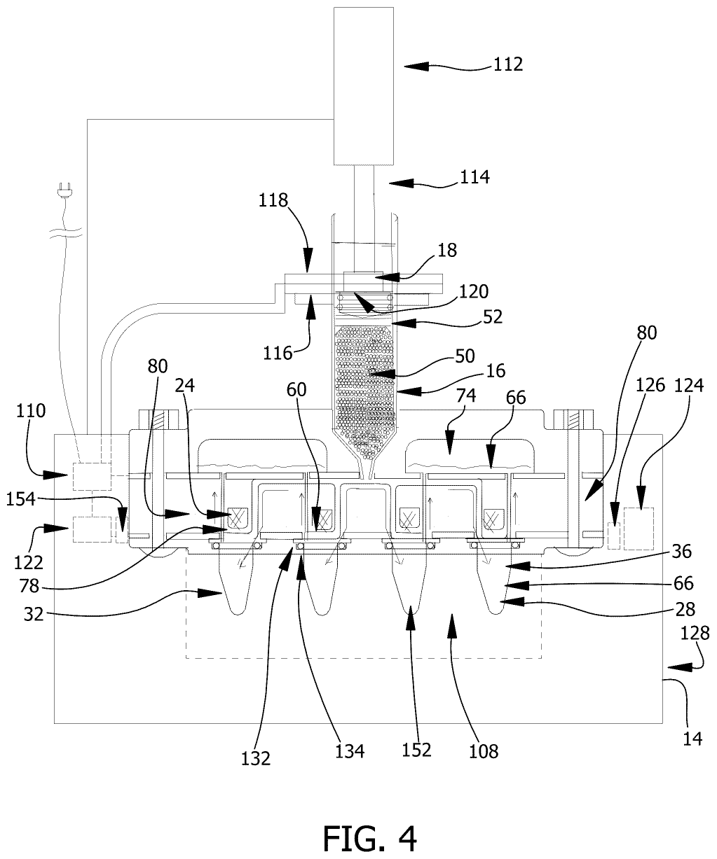

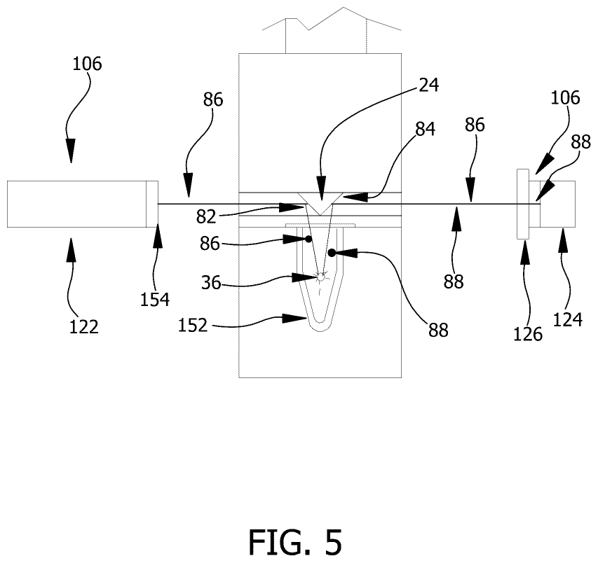

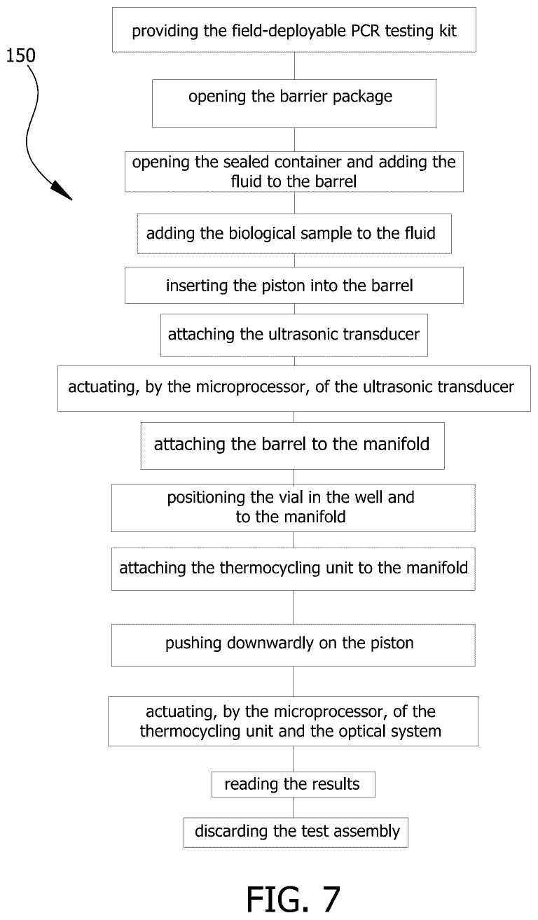

With reference now to the drawings, and in particular to FIGS. 1 through 8 thereof, a new field-deployable test assembly embodying the principles and concepts of an embodiment of the disclosure and generally designated by the reference numeral 10 will be described.

As best illustrated in FIGS. 1 through 8, the field-deployable polymerase chain reaction test assembly 10 generally comprises a barrel 16, a piston 18, a manifold 20, a purification column 22, and a mirror assembly 24. The barrel 16 is configured to receive a fluid 26, such as, but not limited to, water that meets specifications for use in polymerase chain reaction (PCR) testing. The piston 18 is insertable into the barrel 16 to seal the fluid 26 within the barrel 16 after the addition of a biological sample 28 to the fluid 26. The piston 18 may be fitted with one or two O-rings 30 that will sealably engage the barrel 16. A vial 32 having a quantity of PCR reagents 34 positioned therein is selectively attachable to the manifold 20 so that the vial 32 is in fluidic communication with the barrel 16.

The PCR reagents 34 include, but are not limited to, lyophilized up-hill primers, lyophilized downhill primers, fluorescent probes (fluorophores 36), associated fluorescent probe quenchers specific to PCR tests to be run, and all other components required for the PCR test but not contained in the fluid 26. Fluorescent probes 36 for the PCR tests are well known to those skilled in the art of PCR tests and may include, for example, Sybr®Green or similar indicators, target specific DNA fluorophores 36 which become active after their quenching components have been removed during thermocycling, or the like. The vial 32 typically will comprise elastomer having low native fluorescence and may be of black color due to a sensitivity of a PCR test enabled by the field-deployable PCR test assembly 10.

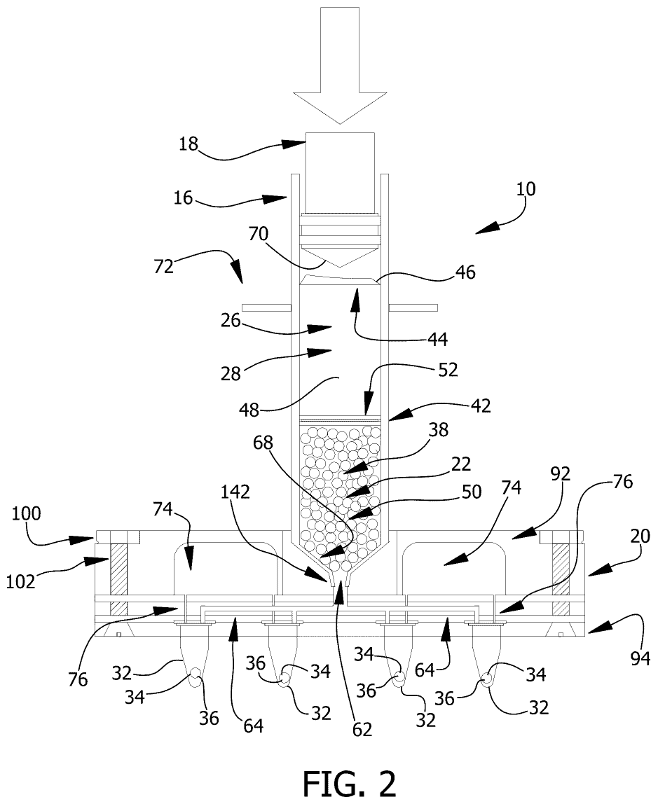

The purification column 22 is positioned within the barrel 16 and substantially occupies a first space 38 proximate to a top face 40 of the manifold 20. As is shown in FIG. 2, the barrel 16 may have a slight internal prominence 42 to position the purification column 22. A marking 44 on the barrel 16 above the purification column 22 defines a predefined fill level 46 for the fluid 26. The fluid 26 substantially occupies a second space 48 between purification column 22 and the marking 44 upon addition of the fluid 26 to the barrel 16. The fluid 26 may contain salts or minerals necessary to support PCR, such as, but not limited to, magnesium chloride. The fluid 26 may further contain reverse transcriptase to change RNA to cDNA.

The purification column 22 includes a separation medium 50 and a circular filter 52. The circular filter 52 is attached to the barrel 16 and is positioned atop the separation medium 50. The circular filter 52 is hydrophobic and has sub-micron pores so that the fluid 26 does not penetrate the circular filter 52 until application of a threshold pressure, approximately 20 psi, by the piston 18. The circular filter 52 is configured to allow passage of dsDNA or cDNA and to substantially exclude materials larger than the dsDNA or cDNA. While the circular filter 52 in the provided Figures does have a circular shape, other shapes may be used which conform to the shape of the barrel 16.

The separation medium 50 is configured to allow passage of the dsDNA or cDNA and to retain impurities, such as, but not limited to, proteins, PCR inhibitors, or the like. Appropriate selection of the separation medium 50 that is suitable for the particular biological sample 28 is required and will be apparent to those skilled in the art of PCR tests.

The purification column 22 eliminates a requirement for traditional extraction and the chemicals required, which may be carcinogenic. Because no traditional extraction chemicals are used to extract the DNA or RNA, their removal or neutralization is not required, as otherwise they would interfere with the PCR test. Additionally, the elution of DNA or RNA from glass fiber or other substrates, as is commonly practiced in PCR preparation protocols, is not required with the field-deployable PCR test assembly 10.

The manifold 20 also has a bottom face 54, a first side face 56, and a second side face 58. The bottom face 54 and the top face 40 have an orifice 60 and an inlet 62 extending therein, respectively. Upon attachment of the vial 32 to the manifold 20, the vial 32 is positioned around and seals the orifice 60. The manifold 20 has a fluid channel 64 positioned therein which is open to the orifice 60 and in fluidic communication with the inlet 62. Thus, the manifold 20 and the vial 32 are in fluidic communication with the barrel 16. The vial 32 is configured to receive a filtrate 66 upon pressurization of the barrel 16.

As is shown in FIG. 2, the barrel 16 has a lower section 68 that extends into the manifold 20 so that the inlet 62 is recessed into the manifold 20. The barrel 16 tapers internally from the top face 40 of the manifold 20 to the inlet 62 so that the lower section 68 is conically shaped. Additionally, a lower end 70 of the piston 18 is conically shaped. As will become apparent below, the lower end 70 of the piston 18 being conically shaped is advantageous in that it is rendered an ultrasonic reflection element.

In one configuration of the field-deployable PCR test assembly 10, as is shown in FIG. 1, the top face 40 of the manifold 20 has a socket 12 extending thereinto. The barrel 16 is configured as a syringe 14 having an outlet end 90, which is selectively insertable into the socket 12 so that the syringe 14 is friction fit to the manifold 20. The outlet end 90 may terminate in a Luer-Slip tip 142 that is complementary to and sealably insertable into the inlet 62. Other configurations, such as the outlet end 90 terminating in a Luer-Lock tip, also are anticipated.

The piston 18 supplies pressure on the fluid 26 to drive the fluid 26 downwardly through the purification column 22 and the manifold 20 and into the vial 32. A pair of tabs 72 may be attached to and extend from the barrel 16 below the predefined fill level 46. The tabs 72 are configured for positioning of fingers of a hand as a thumb of the hand is used to push the piston 18 into the barrel 16. The manifold 20 also has a chamber 74 and a capillary channel 76 positioned therein. The capillary channel 76 extends from the orifice 60 to the chamber 74, thus configuring the chamber 74 to receive air and excess filtrate 66 from the vial 32 as the vial 32 is filled with the filtrate 66. The air naturally rises into the chamber 74 through the capillary channel 76 as the filtrate 66 is introduced under pressure to the vial 32, rendering the manifold 20 a closed system, and thereby decreasing any potential risk of exposure to protect an operator while allowing the PCR test to be performed outside of a containment laboratory. A diameter of the capillary channel 76 promotes capillary action allowing for substantially free passage of air while restricting entry of the filtrate 66. A small differential back pressure (˜0.5 psi) is created which allows the air to pass through the capillary channel 76 and into the chamber 74. When the vial 32 is filled with the filtrate 66 and under sufficient pressure, the capillary channel 76 then allows passage of a small amount of the filtrate 66. The internal pressure in the chamber 74 may be between 25 psi and 60 psi after the piston 18 has been fully depressed.

The manifold 20 has a void 78 and a light channel 80 positioned therein, with the light channel 80 extending bidirectionally from the void 78 to the first side face 56 and the second side face 58. The void 78 is positioned above and opens to the orifice 60. The mirror assembly 24, which is positioned in the void 78, has a first mirror 82 and a second mirror 84. Most typically, the first mirror 82 and the second mirror 84 are primary surface mirrors so as to avoid distorted reflections from a back side of mirror glass of which the first mirror 82 and the second mirror 84 are comprised. The first mirror 82 is positioned to reflect an excitation wavelength of light 86 from the light channel 80 into the vial 32 and the second mirror 84 is positioned to reflect the excitation wavelength of light 86 and fluorescent light 88 that is emitted from the vial 32 into the light channel 80 and toward the second side face 58 of the manifold 20.

The manifold 20 can be manufactured using any number of methods and parts. One nonlimiting example is depicted in the figures wherein the manifold 20 comprises an upper piece 92 incorporating the chamber 74, a lower plate 94, an upper gasket 96, and a lower gasket 98. The upper gasket 96 and the lower gasket 98 are formed to allow passage of the fluid 26 through the fluid channel 64 and the capillary channel 76. The manifold 20, as is shown in the figures, is attached together using machine nuts 100 and machine screws 102, although other forms of mechanical attachment may be used such as, but not be limited to, rivets, snap-together ridged thermoplastic components, adhesive bonding, or thermal bonding afforded by laser or ultrasonic bonding, or any means of reliably bonding components. Furthermore, the manifold 20 could also be 3D printed or formed by injecting molding.

The field-deployable PCR test assembly 10 also includes an ultrasonic transducer 104, an optical system 106, a thermocycling unit 108, a microprocessor 110, and may include a linear actuator 112. The thermocycling unit 108 is in thermal communication with the vial 32 and is configured to thermocycle the PCR reagents 34 and the filtrate 66 posited within the vial 32. The thermocycling unit 108 has a well 152 extending thereinto and is configured for insertion of the vial 32 so that the vial 32 is in thermal communication with the thermocycling unit 108. The thermocycling unit 108 is selectively attachable to the bottom face 54 of the manifold 20 whereupon the vial 32 is sealably attached to the manifold 20 for sealing the orifice 60. The sealing may be achieved with a flange 132 and ring gasket 134. The vial 32 is configured to receive the filtrate 66 upon pressurization of the barrel 16.

As is shown in FIGS. 2-4, the vial 32 may be one of a plurality of vials 32, with each vial 32 being in fluidic communication with the barrel 16. The well 152, in such a configuration, is one of a plurality of wells 152 that extends into the thermocycling unit 108 and the optical system 106 is one of a plurality of optical systems 106. Thus, each well 152 is occupied by a respective vial 32 and each vial 32 is paired with a respective optical system 106. In this manner, with each vial 32 containing a quantity of PCR reagents 34 specific to a particular strand of DNA potentially present in the biological sample 28, a plurality of PCR tests can be performed simultaneously. For example, with four vials 32 each containing a PCR reagent 34 specific to a type of malaria, a subject can be tested once to detect any of four different types of malaria.

The thermocycling unit 108 is a standard component used in PCR tests and alternately heats and cools the fluid 26 filtrate 66 in the vial 32 in repeated cycles designed to produce DNA amplification of a targeted segment of DNA when it is present. Such thermocycling units 108 are well known to those skilled in the art of PCR tests. However, the thermocycling unit 108 of the present invention differs from the prior art in that spacing of the wells 152 is closer and in that the thermocycling unit 108 is comprised of silver or copper, thereby facilitating heat transfer and shortening the required time for thermocycling. Typically, the upper temperature of each thermocycle is set at 95 degrees C. As the temperature drops through 75-70 degrees C., elongation of the DNA strands occurs by use of a special polymerase. Pausing the temperature at this level is not required. The bottom temperature relies on the molecular length of the primers and may be set between 65 degrees and 50 degrees C.

The ideal yield of duplicated targets formed by PCR amplification with thermocycling follows the simple mathematical expression of 2ⁿ (2 raised to the power of n), where n is equal to the number of thermal cycles performed by the thermocycling unit 108. The number of thermal cycles can be significantly reduced with the current invention as it gathers meaningful signal information from within what was previously considered unreadable “background noise”, as is discussed further below.

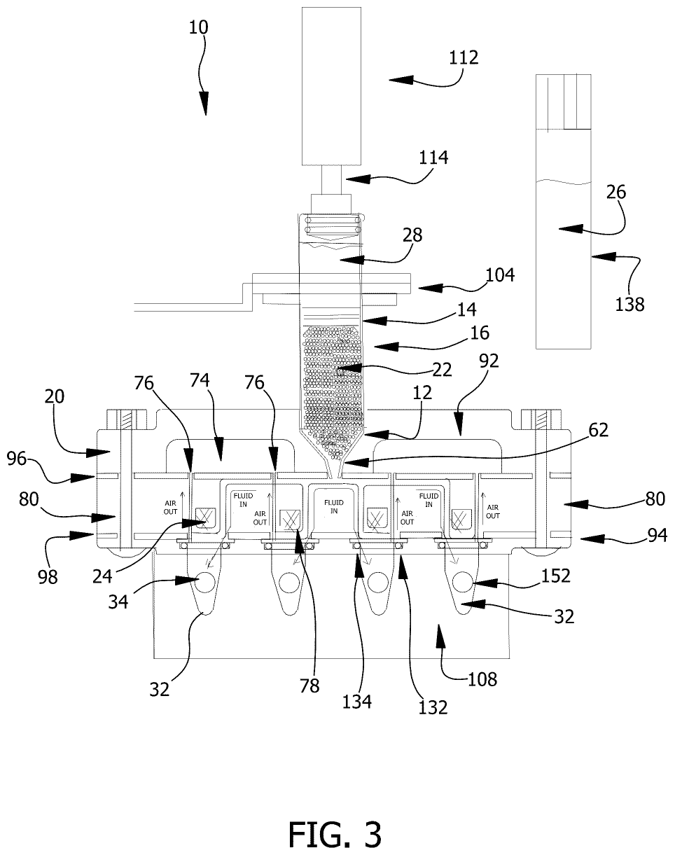

The ultrasonic transducer 104 is in communication with the barrel 16 and is configured to provide transient cavitation pressure on the fluid 26 to release DNA or RNA from the biological sample 28 in the fluid 26. The ultrasonic transducer 104 is selectively attachable to the barrel 16 below the marking 44 and above the purification column 22. By way of example and as depicted in FIGS. 3-5, the ultrasonic transducer 104 may comprise a piezoelectric device 116, which is flat and ring shaped, and is attached to an aluminum resonating disk 118. The piezoelectric device 116 transforms electronic energy into ultrasound waves at approximately 40 kHz, with the ultrasound waves being mechanically propagated by the aluminum resonating disk 118 to the fluid 26. Sonication may last from a few seconds to a few minutes and is used to release DNA and/or RNA from the biological sample 28.

The ultrasonic transducer 104 has a central lumen 120 so that the aluminum resonating disk 118 can be force-fit snugly to the barrel 16. The barrel 16 may taper slightly to enhance the force-fit, allowing the aluminum resonating disk 118 to slide along the barrel 16 until it binds adjacent to the second space 48 in which the fluid 26 is positioned. The ultrasonic transducer 104 also may rest upon the pair of tabs 72, if included, upon insertion of the barrel 16 into the central lumen 120. Effectively, the tabs 72 establish a limit to a lower position of the ultrasonic transducer 104. The ultrasonic energy creates cavitation bubbles in the fluid 26 which arise and then spontaneously collapse under the action of the ultrasonic sound waves. Collapse of the cavitation bubbles releases high-speed microjets, at about 400 mph, of the fluid 26 and a photon. This process hydraulically abrades surfaces of the biological sample 28, such as cellular structures and external parts of viruses and bacteria, thereby releasing DNA and/or RNA from the biological sample 28.

The optical system 106 emits the excitation wavelength of light 86 into the vial 32 and is configured to read the fluorescent light 88 that is emitted from the vial 32 to indicate the presence of dsDNA within the vial 32. More specifically, the optical system 106 comprises an emitter 122 and a detector 124. The emitter 122 emits the excitation wavelength of light 86 into the light channel 80 and into the vial 32 to excite the fluorophore 36 indicating replication of a particular dsDNA or a particular segment of dsDNA, which is a PCR target to be replicated when present, whereupon the fluorophore 36 emits the fluorescent light 88 through the light channel 80 to the detector 124. The emitter 122 may comprise various light emitting devices including, for example, a compact laser, a light emitting diode, or the like. The detector 124 may comprise a high sensitivity solid-state low-light-sensitive photon receiver, a solid-state photomultiplier device, an avalanche diode, or other similar low light detector.

Typically, an optical low pass filter 126 is attached to the detector 124 and is positioned between the detector 124 and the second side face 58 of the manifold 20. The optical low pass filter 126 is configured to filter out the excitation wavelength of light 86 that exits the light channel 80 from the second side face 58. Additionally, an optical notch filter 154 typically is attached to the emitter 122 and is positioned between the emitter 122 and the first side face 56 of the manifold 20. The optical notch filter 154 is configured to filter out light that is not of the excitation wavelength of light 86.

The emitter 122 and the detector 124 may be selectively attachable to the first side face 56 and the second side face 58, respectively. Alternatively, the emitter 122 and the detector 124 could be positioned adjacent to the first side face 56 and the second side face 58, respectively, and in alignment with the light channel 80.

The fluorescent light 88 that is emitted from the vial 32 is shifted longer according to the physics of fluorescent materials by 20 nanometers, or more, relative to the excitation wavelength of light 86. The excitation wavelength of light 86 traveling through the light channel 80 typically is reflected at an angle of 95°-97° from the first mirror 82 of the mirror assembly 24 into the vial 32 where it can excite the fluorophore 36. The fluorescent light 88, which is emitted by the fluorophore 36 in the vial 32, is reflected at an angle of 95°-97° from the second mirror 84 of the mirror assembly 24 back into the light channel 80 and through the optical low pass filter 126 to the detector 124. The intensity of the fluorescent light 88 that is emitted from the vial 32 is measured at the end of each thermal cycle with measurable increases being detected beginning at the second thermal cycle. The intensity of the fluorescent light 88 that is being measured by the detector 124 consistently increases as more dsDNA is created via PCR replication.

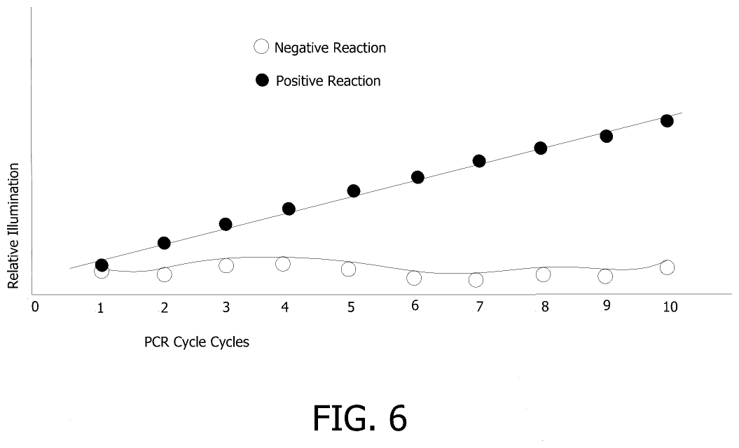

The limit of detection for the optical system 106 of the present invention is capable of measuring increases in fluorescent light 88 that is emitted throughout what has been termed “background” emission levels for most Real-time (qPCR) fluorescent-reading thermal cyclers, which pass through several cycles until their limit of detection is reached. Thus, the present invention allows reading of the PCR binary expansion of dsDNA at early cycles usually considered unreadable because of background noise, resulting in not only exceptionally fast sample preparation but very quick data to confirm positive or negative samples in as little as 10 thermal cycles. FIG. 6 represents readings from the optical system 106, which is log-based, for the readings taken after each of the first 10 cycles of the PCR test using the present invention. There is a clear separation at the second cycle between a positive reading (solid circles) and a background/negative reading (open circle).

The microprocessor 110 is in communication with the ultrasonic transducer 104, the thermocycling unit 108, the emitter 122, the detector 124, and, if included, the linear actuator 112. The microprocessor 110 is programmed to execute the PCR test to identify at least one particular DNA strand present in the biological sample 28. The field-deployable PCR test assembly 10 enables onsite completion of the PCR test in less than thirty minutes. The microprocessor 110 also is enabled to selectively actuate the linear actuator 112, if included. The linear actuator 112 comprises a pushrod 114 that is in alignment with, or alignable with, the piston 18. When the linear actuator 112 is actuated by the microprocessor, the pushrod 114 pushes the piston 18 into the barrel 16. Such linear actuators 112 are well known to those skilled in the art of dispensing from syringes and may utilize position sensors, timers, or the like for positioning of the pushrod 114.

The microprocessor 110 typically is preprogrammed for a particular PCR test so that the ultrasonic transducer 104 is actuated for a first prescribed time period, the piston 18 is pushed downwardly for a second prescribed time period (either manually or using the linear actuator 112), and the thermocycling unit 108 is actuated to perform a predetermined number of thermocycles having predetermined durations.

The present invention anticipates the emitter 122, the detector 124, and the thermocycling unit 108 being selectively attachable to the manifold 20 using a variety of attachment means, such as, but not limited to, snap-fit connections, slid locking connections, push lock mechanisms, or the like. Additionally, the thermocycling unit 108, the emitter 122, the detector 124, and the microprocessor 110 could comprise a unitary structure 128 having a recess 130 into which the manifold 20 would fit. With the manifold 20 being a closed system, the electronics can be used without contamination and can potentially be used repeatedly with fresh test assemblies 10.

The present invention also anticipates a transceiver being operationally engaged to the microprocessor 110 allowing for wireless control and receipt of test results from an electronic device, such as a smartphone, a laptop computer, or the like. Additionally, while the figures depict the electronics components as connecting to a power source, the use of battery power also is anticipated.

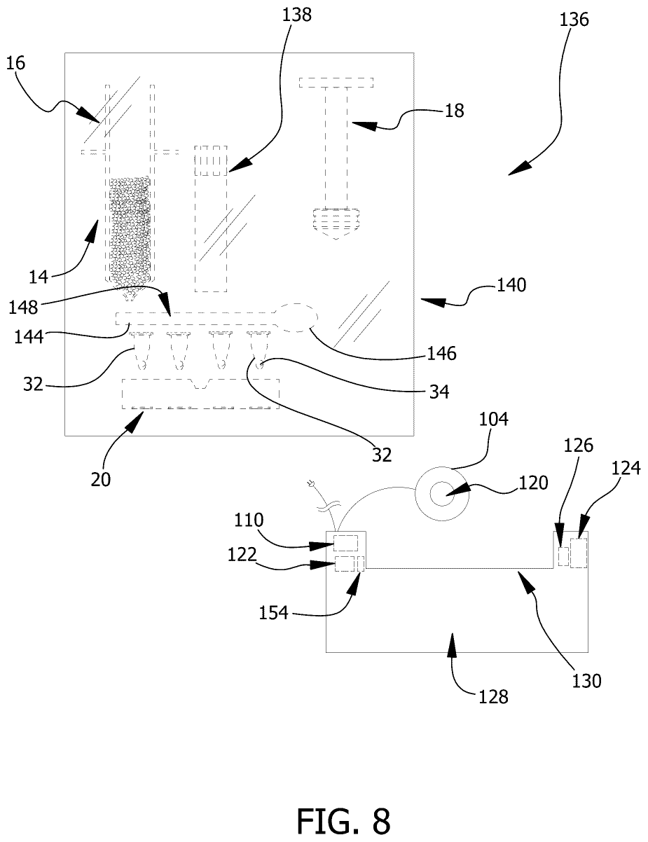

As is depicted in FIG. 8, the present invention also includes a field-deployable PCR test kit 136, which comprises the barrel 16, the manifold 20, the piston 18, and the vial 32, as described above, and a sealed container 138 containing a predetermined amount of the fluid 26. A typical volume of the sealed container 138 is 2.0 mL and it may be formatted as a centrifuge tube. The barrel 16, the manifold 20, the piston 18, the vial 32, and the sealed container 138 are enveloped within a barrier package 140 to maintain the same in a contaminant-free environment. The barrier package 140 typically will comprise a foil and polymer pouch or similar packaging.

A stemmed swab 144 (or equivalent) to collect the biological sample 28 may be included in the barrier package 140. Introduction of the biological sample 28 can be performed using the stemmed swab 144 to collect, for example, mucus, cells, pathogens, or the like by swabbing a desired area with a tuft 146 of the stemmed swab 144 and then twirling a stem 148 of the stemmed swab 144 several times while the tuft 146 is submerged in the fluid 26. Properly prepared biological samples 28 of a variety of biological forms may be judiciously introduced into the fluid 26 using non-contaminating techniques familiar to those skilled in the art of PCR testing. The field-deployable PCR test kit 136 is intended to enable onsite completion of the PCR test in less than thirty minutes. The field-deployable PCR test kit 136 also may include the thermocycling unit 108, the microprocessor 110, and the linear actuator 112.

In use, the field-deployable PCR test kit 136 enables a method for completing a PCR test in less than thirty minutes and can be performed outside of a laboratory 150. The method 150 entails provision of the field-deployable PCR test kit 136, as per the specification above. After opening the barrier package 140, the sealed container 138, the piston 18, the barrel 16, and the manifold 20 are removed. The fluid 26 and the biological sample 28 are added to the barrel 16 at which point the piston 18 is partially inserted into the barrel 16. The ultrasonic transducer 104 is attached to the barrel 16 and actuated for the first prescribed time period to generate ultrasound waves to release DNA and/or RNA from the biological sample 28. The barrel 16 and the vial 32 being attached to the to the manifold 20, the piston 18 then is pushed downwardly for the second prescribed time period to motivate the fluid 26 through the purification column 22 so that the filtrate 66 passes through the manifold 20 to the vial 32. The optical system 106 then is actuated to indicate the presence of DNA within the vial 32 as the thermocycler performs DNA replicating cycles.

With the field-deployable PCR test kit 136 including the thermocycling unit 108 and the microprocessor 110, the method 150 includes the additional steps of positioning the vial 32 in the well 152 upon completion of sonication, attaching the thermocycling unit 108 to the bottom face 54 of the manifold 20, actuating, by the microprocessor 110, of the thermocycling unit 108, thermocycling of the PCR reagents 34 and the filtrate 66, and actuating, by the microprocessor 110, of the optical system 106. Should the barrel 16 be configured as a syringe 14, the method includes an additional step of attaching the syringe 14 to the manifold 20 upon completion of sonication.

The field-deployable PCR test kit 136 is designed to significantly de-skill the PCR process so that it can be used by technicians with limited PCR testing experience. The field-deployable PCR test kit 136 requires no ancillary equipment, such as a microcentrifuge, pipetting equipment, or the like. The intent of the field-deployable PCR test kit 136 is to provide a quicker point-of-care or remote site PCR test providing either a positive or a negative result. It is designed to be used where performing a conventional PCR test may require more time than is expedient for determining either a positive or a negative result. The field-deployable PCR test kit 136 enables the proper treatment of a patient to begin more quickly than is possible with conventional PCR, which may require several hours to days before the test result is known.

The result obtained from the field-deployable PCR test kit 136 is available to a user much quicker relative to traditional means, with traditional PCR machine testing taking two or more hours. Moreover, most PCR equipment today is distal to where a biological sample 28 is collected, so shipping and processing may take days to weeks. It currently is not possible to provide prompt results that might enable a person who is infected with a disease, but who is asymptomatic, to make knowledgeable decisions to avoid infecting others. The field-deployable PCR test kit 136 has the capacity to reduce the spread of disease from asymptomatic infected persons by providing necessary information.

Current PCR preparation steps may take more than an hour. A typical qPCR device will require up to an hour for preparation of the biological sample 28 and about one to two minutes for each thermocycle, or approximately 40 to 120 minutes for thermocycling. The use of the manifold 20 described in this patent may shorten the prep-time to around one to two minutes and the optical system of the field-deployable PCR test kit 136 described herein can shorten the thermal cycling time to around ten minutes. Thus, the field-deployable PCR test kit 136 can provide a result (either positive or negative) in terms of minutes, most typically less than 30 minutes and often less than 15 minutes.

PCR testing during the Covid-19 SARS virus pandemic typically took days to weeks to confirm the presence of the SARS virus, thereby allowing an infected person to expose others before the presence of the virus was confirmed. Antibody tests for viral infections may not be dependable early-stage indicators for a disease, particularly soon after infection when the antibody titer is low and not yet detectable with an antibody test. However, the virus already is reproducing at very rapid rates and can be detected using a PCR test. Although a patient may not yet feel sick, they still can spread the disease to others and the infection may be well established before the patient is symptomatic. Antibody levels also are limited in determining if a virus has been eliminated from a host. The lack of PCR machines at points-of-care to provide a definitive result in minutes, while a patient waits, can contribute to the inadvertent spread of a high morbidity and/or a high mortality infection and may result in a pandemic.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A field-deployable polymerase chain reaction (PCR) test assembly comprising:
a barrel configured for receiving a fluid;
a manifold being in fluidic communication with the barrel such that the fluid flows downwardly into the manifold from the barrel;
a piston being insertable into the barrel, the piston sealing the fluid within the barrel and pressuring the fluid;
a vial being attachable to the manifold such that the vial is in fluidic communication with the barrel;
a quantity of PCR reagents being positioned in the vial;
a purification column being positioned within the barrel, the piston supplying pressure on the fluid to drive the fluid through the purification column and into the vial;
an ultrasonic transducer being in communication with the barrel and being configured to provide transient cavitation in the fluid to release DNA or RNA from a biological sample in the fluid; and
an optical system emitting an excitation wavelength of light into the vial and being configured for reading fluorescent light emitted from the vial indicating the presence of DNA within the vial.

2. The field-deployable PCR test assembly of claim 1, further including:
the manifold having a fluid channel positioned therein, the manifold having top and bottom faces, the manifold having first and second side faces, the bottom face having an orifice extending therein and being in fluidic communication with the fluid channel, the top face having an inlet extending therein and being in fluidic communication with the fluid channel, the manifold having a void and a light channel positioned therein, the void being positioned above and being open to the orifice;
a thermocycling unit having a well extending thereinto, the well being configured for insertion of the vial such that the vial is in thermal communication with the thermocycling unit, the thermocycling unit being selectively attachable to the bottom face of the manifold whereupon the vial is sealably attached to the manifold for sealing the orifice, wherein the vial is configured for receiving a filtrate upon pressurization of the barrel and wherein the thermocycling unit is configured for thermocycling of the PCR reagents and the filtrate positing within the vial;
the optical system comprising an emitter and a detector, wherein the emitter emits the excitation wavelength of light into the light channel and into the vial to excite a fluorophore indicating replication of a target DNA, whereupon the fluorophore emits the fluorescent light through the light channel to the detector;
a microprocessor being in communication with the ultrasonic transducer, the thermocycling unit, the emitter, and the detector and being programmed to execute a PCR test to identify at least one particular DNA stand present in the biological sample; and
wherein the field-deployable PCR test assembly enables onsite completion of a PCR test in less than thirty minutes.

3. The field-deployable PCR test assembly of claim 2, further including:
the light channel extending bidirectionally from the void to the first and second side faces of the manifold;
the emitter and the detector being selectively attachable to the first and second side faces, respectively; and
a mirror assembly being positioned in the void, the mirror assembly having:
a first mirror positioned for reflecting the excitation wavelength of light from the light channel into the vial; and
a second mirror positioned for reflecting the excitation wavelength of light and the fluorescent light emitted from the vial into the light channel and toward the second side face of the manifold.

4. The field-deployable PCR test assembly of claim 3, further including:
the emitter comprising a compact laser or a light emitting diode;
an optical notch filter being attached to the emitter and positioned between the emitter and the first side face of the manifold, wherein the optical notch filter is configured for filtering out light not of the excitation wavelength;
the detector comprising a high sensitivity solid-state low-light-sensitive photon receiver; and
an optical low pass filter being attached to the detector and positioned between the detector and the second side face of the manifold, wherein the optical low pass filter is configured for filtering out the excitation wavelength of light exiting the light channel at the second side face.

5. The field-deployable PCR test assembly of claim 2, further including:
the top face of the manifold having a socket extending thereinto; and
the barrel being configured as a syringe having an outlet end, the outlet end being selectively insertable into the socket such that the syringe is friction fit to the manifold.

6. The field-deployable PCR test assembly of claim 1, further including a marking on the barrel above the purification column, the marking defining a predefined fill level for the fluid, the ultrasonic transducer being selectively attachable to the barrel below the marking and above the purification column.

7. The field-deployable PCR test assembly of claim 6, further including a pair of tabs being attached to and extending from the barrel below the predefined fill level, wherein the tabs are configured for positioning of fingers of a hand as a thumb of the hand is used to push the piston into the barrel, the ultrasonic transducer being selectively attachable to the barrel such that the ultrasonic transducer rests upon the pair of tabs.

8. The field-deployable PCR test assembly of claim 6, wherein the purification column substantially occupies a first space proximate to the top face of the manifold, the purification column comprising:
a separation medium;
a circular filter being attached to the barrel and positioned atop the separation medium, the circular filter being hydrophobic and having sub-micron pores such that the fluid does not penetrate the circular filter until application of a threshold pressure by the piston, wherein the circular filter is configured for allowing passage of DNA and for substantially excluding materials larger than the DNA and wherein the separation medium is configured for separating the DNA from impurities; and
the fluid substantially occupying a second space between the purification column and the marking upon addition of the fluid to the barrel.

9. The field-deployable PCR test assembly of claim 2, further including the manifold having a chamber and a capillary channel positioned therein, the capillary channel extending from the orifice to the chamber, wherein the chamber is configured for receiving air and excess filtrate from the vial as the vial is filled with the filtrate.

10. The field-deployable PCR test assembly of claim 2, further including a linear actuator, the linear actuator comprising a pushrod in alignment with the piston, the linear actuator being in communication with the microprocessor such that the microprocessor is enabled for selectively actuating the linear actuator for pushing the piston into the barrel.

11. The field-deployable PCR test assembly of claim 1, further including the vial comprising elastomer having low native fluorescence.

12. The field-deployable PCR test assembly of claim 2, further including:
the barrel having a lower section extending into the manifold such that the inlet is recessed into the manifold; and
the barrel tapering internally from the top face of the manifold to the inlet such that the lower section is conically shaped.

13. The field-deployable PCR test assembly of claim 1, wherein:
the vial is one of a plurality of vials, each vial being in fluidic communication with the barrel via the manifold, each vial having positioned therein a quantity of PCR reagents specific to a particular strand of DNA potentially present in the biological sample; and
the optical system is one of a plurality of optical systems, each optical system being paired with a respective one of the vials of the plurality of vials.

14. A field-deployable polymerase chain reaction (PCR) test kit comprising:
a barrel configured for receiving a fluid;
a manifold being in fluidic communication with the barrel such that the fluid flows downwardly into the manifold from the barrel;
a piston being insertable into the barrel, the piston sealing the fluid within the barrel and pressuring the fluid;
a vial being attachable to the manifold such that the vial is in fluidic communication with the barrel;
a quantity of PCR reagents being positioned in the vial;
a purification column being positioned within the barrel, the piston supplying pressure on the fluid to drive the fluid through the purification column and into the vial;
a sealed container containing a predetermined amount of the fluid;
a barrier package enveloping the barrel, the manifold, the piston, the vial, and the sealed container, wherein the barrier package is configured for maintaining the barrel, the manifold, the piston, the vial, and the sealed container in a contaminant-free environment;
an ultrasonic transducer being attachable to the barrel such that the ultrasonic transducer is in communication with the barrel and configured to provide transient cavitation pressure on the fluid to release DNA or RNA from a biological sample in the fluid; and
an optical system emitting light into the vial and being configured for reading fluorescent light emitted from the vial indicating the presence of DNA within the vial.

15. The field-deployable PCR test kit of claim 14, wherein the PCR test kit enables onsite completion of a PCR test in less than thirty minutes.

16. A method for using a field-deployable polymerase chain reaction (PCR) test kit to complete a PCR test in less than thirty minutes outside of a laboratory, the method comprising the steps of:
providing the field-deployable PCR test kit comprising:
a barrel configured for receiving a fluid;
a manifold being in fluidic communication with the barrel such that the fluid flows downwardly into the manifold from the barrel;
a piston being insertable into the barrel, the piston sealing the fluid within the barrel and pressuring the fluid;
a vial being attachable to the manifold being such that the vial is in fluidic communication with the barrel;
a quantity of PCR reagents being positioned in the vial;
a purification column being positioned within the barrel, the piston supplying pressure on the fluid to drive the fluid through the purification column and into the vial;
a sealed container containing a predetermined amount of the fluid;
a barrier package enveloping the barrel, the manifold, the piston, the vial, and the sealed container, wherein the barrier package is configured for maintaining the barrel, the manifold, the piston, the vial, and the sealed container in a contaminant-free environment;
an ultrasonic transducer being attachable to the barrel such that the ultrasonic transducer is in communication with the barrel and configured to provide transient cavitation pressure on the fluid to release DNA or RNA from a biological sample in the fluid; and
an optical system emitting light into the vial and being configured for reading fluorescent light emitted from the vial indicating the presence of dsDNA within the vial;
opening the barrier package;
opening the sealed container and adding the fluid to the barrel;
adding the biological sample to the fluid;
inserting the piston into the barrel;
attaching the ultrasonic transducer to the barrel;
actuating the ultrasonic transducer for a first prescribed time period for generating ultrasound waves to release DNA and/or RNA from the biological sample;
pushing downwardly on the piston for a second prescribed time period for motivating the fluid through the purification column such that a filtrate passes to the vial; and
actuating the optical system.

17. The method of claim 16, wherein:
the field-deployable PCR test kit comprises:
a thermocycling unit having a well extending thereinto, the well being configured for insertion of the vial such that the vial is in thermal communication with the thermocycling unit, the thermocycling unit being selectively attachable to the bottom face of the manifold whereupon the vial is sealably attached to the manifold for sealing the orifice, wherein the vial is configured for receiving a filtrate upon pressurization of the barrel and wherein the thermocycling unit is configured for thermocycling of the PCR reagents and the filtrate positing within the vial; and
a microprocessor, the microprocessor being in communication with the ultrasonic transducer, the thermocycling unit, and the optical system and being programmed to execute a PCR test to identify at least one particular DNA stand present in the biological sample;
the method includes the additional steps of:
actuating, by the microprocessor, of the ultrasonic transducer;
positioning the vial in the well upon completion of sonication;
attaching the thermocycling unit to the bottom face of the manifold;
actuating, by the microprocessor, of the thermocycling unit;
thermocycling of the PCR reagents and the filtrate; and
actuating, by the microprocessor, of the optical system.

18. The method of claim 16, further including:
a top face of the manifold having a socket extending thereinto;
the barrel being configured as a syringe having an outlet end, the outlet end being selectively insertable into the socket such that the syringe is friction fit to the manifold; and
the method including the additional step of attaching the syringe to the manifold upon completion of sonication.
